(19) United States
(12) Patent Application Publication
(10) Pub. No.: US 2009/0131360 A1
(43) Pub. Date: May 21, 2009

(54) Tripartite RNAi constructs

(75) Inventors: Tod M. Woolf, Sudbury, MA (US); Pamela A. Pavco, Longmont, CO (US); William Salomon, Framingham, MA (US); Dmitry Samarsky, Westborough, MA (US); Nassim Usman, Palo Alto, CA (US); Rick Wagner, Cambridge, MA (US)

Correspondence Address: ROPES & GRAY LLP, PATENT DOCKETING 39/41, ONE INTERNATIONAL PLACE, BOSTON, MA, 02110-2624, US

(73) Assignee: Rxi Pharmaceuticals, Corp.

(21) Appl. No.: 12/286,896

(22) Filed: Oct. 2, 2008

Related U.S. Application Data
(60) Provisional application No. 60/976,855, filed on Oct. 2, 2007.
(60) Provisional application No. 60/976,858, filed on Oct. 2, 2007.

(51) Int. Cl. A61K 31/7105; C07H 21/02; C07K 2/00; C12N 5/06; C07K 16/18

(52) U.S. Cl. 514/44; 536/24.5; 530/300; 530/391.1; 435/375

(57) ABSTRACT

The present invention provides compositions and methods for inhibiting expression of a target gene in a cell. The process comprises introduction of double-stranded tripartite RNAi constructs into the cells and reducing the expression of the corresponding messenger RNA in the cells. The constructs, which may be packaged in or delivered as sequestered RNAi constructs, differ from the canonical siRNA in that they comprise a tripartite structure which follows the general formula of having (1) an RNAi core (either native or abbreviated), (2) one or more terminal moieties attached to the RNAi core and optionally (3) a linker between the RNAi core and the terminal moiety. Once packaged into sequestration vehicles, the constructs are activated for gene regulation by the application of certain forms of energy

### REFERENCE TO RELATED APPLICATIONS

This application claims the benefit of the filing dates under 35 U.S.C. § 119(e) of U.S. Provisional Application Nos. 60/976,855 and 60/976,858, both filed on Oct. 2, 2007, the entire contents of which are incorporated herein by reference.

### BACKGROUND OF THE INVENTION

Discovered over a decade ago, RNA interference or “RNAi” is an evolutionarily conserved gene silencing phenomenon resulting from exposure to double-stranded RNA (dsRNA). The term has come to generalize all forms of gene silencing involving dsRNA leading to the sequence-specific reduction of endogenous targeted mRNA levels.

Efforts to characterize the mediators of RNAi have resulted in the discovery that 21-23 base pair dsRNA constructs reduce gene expression. These dsRNA, which are now commonly known as short interfering RNAs or “siRNAs,” were shown to be generated by an RNase III-like processing reaction from long dsRNA. Chemically synthesized 21-mer siRNA duplexes having 19 complementary central base pairs with overhanging 3′ ends of DNA (dTdT) are now considered the “canonical siRNA construct and mediate efficient target RNA cleavage (Fire et al., Nature 391: 806-811, 1998; Elbashir et al., Genes Dev. 15: 188-200, 2001; Elbashir et al., Nature 411: 494-498, 2001).

In spite of the tremendous progress in the field, with canonical siRNAs being the construct of choice for target knockdown in vitro, there remain several technological hurdles in the development of RNAi-based therapeutics. These include development of effective delivery vehicles, optimization of pharmacokinetic properties including toxicity and bioavailability, reduction of off-target effects including interference with other RNA-based pathways such as those involving miRNA and elimination of interference with the innate immune response.

### SUMMARY OF THE INVENTION

The present invention provides compositions and methods for inhibiting expression of a target gene in a cell. The process comprises introduction of double-stranded RNAi constructs into the cells and reducing the expression of the corresponding messenger RNA in the cells. The RNAi constructs of the present invention differ from the canonical siRNA in that they comprise a tripartite structure which follows the general formula of having (1) an RNAi core (either native or abbreviated), (2) one or more terminal moieties attached to the RNAi core, and, optionally, (3) a linker between the RNAi core and the terminal moiety.

The RNAi cores of the present invention comprise either a native or abbreviated form. In the native form the RNAi core comprises a blunt-ended double-stranded RNA (dsRNA) where each strand is between 25-30, preferably 25-27 and more preferably 25 nucleotides in length. The abbreviated RNAi core, based on the native RNAi core, is configured such that a contiguous portion of the native core is substituted with a functional moiety. The effect of the substitution is to reduce the overall size of the dsRNA portion of the RNAi core and to impart to the construct a beneficial feature, property or characteristic. The RNAi cores of the present invention may also comprise chemical modifications but the extent of the modifications is minimal involving no more than 2 types of modifications at no more than 10 sites along the RNAi core.

The present invention also provides compositions and methods for spatial and/or temporal inhibition of expression of a target gene in a cell. The process comprises introduction of sequestered double-stranded RNAi constructs into the cells and reducing the expression of the corresponding messenger RNA in the cells. The RNAi constructs, or components thereof, of the present invention are packaged in or delivered as sequestered constructs. The sequestration vehicles of the present invention include, but are not limited to, liposomes, nanotransporters, composites, metal complexes, polymers or biopolymers such as hydroxyapatite, nanoparticles, microparticles or any other vehicle considered useful for the targeted delivery of nucleic acid constructs. While many nucleic acid delivery vehicles are known in the art, the sequestration vehicles of the present invention differ in that the RNAi agents of the sequestration vehicles are activated via the application of energy, either directly to the sequestration vehicle or to a system comprising a sequestration vehicle, such as a cell, tissue or organism.

Thus, one aspect of the invention provides an RNAi construct comprising: (a) an RNAi core comprising a blunt-ended double-stranded RNA (dsRNA) with or without a portion of the RNAi core substituted with a non-nucleic acid based functional moiety, the RNAi core consisting of a sense strand and an antisense strand, each strand being 25-30 nucleotides in length, wherein the sense strand is chemically modified, and wherein the antisense strand is at least partially complementary to and hybridizes with a transcript from a target gene, and, (b) one more terminal moieties, preferably, each independently selected from: a bimodal partner, a carrier mimic, a membrane intercalator, a lipophilic molecule, a reporter molecule, a vitamin, a drug, a toxin, a polymer, a peptide, an antibody or a functional fragment, a carbohydrate, a nucleic acid cleaving complex, a metal chelator, an intercalator, a crosslinking agent, a cholesterol, a lipid moiety, a phospholipid, biotin, phenazine, a folate, phenanthridine, anthraquinone, acridine, a fluorescein, rhodamine, a coumarin, a dye, an active drug substance, or a group that enhances a pharmacodynamic property selected from construct uptake, construct resistance to degradation, and/or sequence-specific hybridization with the transcript, wherein the RNAi core is linked to the one or more terminal moieties either directly or through one or more linkers.

In certain embodiments, the bimodal partner is an antisense, a ribozyme, an RNAi molecule which antagonizes the function of a gene other than the target gene.

In certain embodiments, the sense strand is chemically modified by the incorporation of modified oligonucleotide backbones containing a phosphorous atom.

In certain embodiments, the sense strand is chemically modified by the incorporation of 2′ substituent groups at either or both of the 5′ and 3′ ends.

In certain embodiments, the sense strand is chemically modified by the incorporation of four identical 2′ substituent groups in each terminus.

In certain embodiments, the sense strand is chemically modified by the incorporation of between three to five 2′-O-methyl groups at the 5′ end and the 3′ end.

In certain embodiments, the sense strand is chemically modified by the incorporation of four 2′-O-methyl groups at the 5′ end and the 3′ end.

In certain embodiments, each strand of the blunt-ended dsRNA is 25-27 nucleotides in length.

In certain embodiments, each strand of the blunt-ended dsRNA is 25 nucleotides in length.

In certain embodiments, a portion of the RNAi core is substituted with the functional moiety which imparts the RNAi construct a feature, property, or characteristic.

In certain embodiments, the functional moiety improves cellular distribution, bioavailability, activity, resistance to degradation, sequence-specific hybridization, metabolism, excretion, permeability, and/or cellular uptake of the RNAi construct.

In certain embodiments, the blunt-ended dsRNA is 25 nucleotides in length, and wherein about 15-20 contiguous nucleotides of the dsRNA is retained while the remaining 5-10 nucleotides of the dsRNA are substituted with the functional moiety.

In certain embodiments, the blunt-ended dsRNA retains about 7, 8, 9, or 10 contiguous nucleotides.

In certain embodiments, the functional moiety is a protein, a peptide, a carbohydrate, or a lipid.

In certain embodiments, the protein or the peptide comprises a segment, a region, a domain, or a portion of a RISC(RNAi Induced Silencing Complex) protein, a RISC complex comprising the RISC protein, or a protein associated with the RISC complex.

In certain embodiments, the segment, the region, the domain, or the portion improves the affinity of the RNAi construct to the RISC complex.

In certain embodiments, the functional moiety associates with the translation machinery of a cell.

In certain embodiments, the functional moiety associates with a polyribosome or a ribosomal subunit.

In certain embodiments, the transcript is a protein-coding mRNA.

In certain embodiments, the transcript is a non-protein-coding RNA sequence.

In certain embodiments, the one or more terminal moieties comprise one or more chemical modifications, protecting groups, and/or substituent groups.

In certain embodiments, the one or more terminal moieties provide a charged environment to sequester the RNAi core from a cellular milieu.

In certain embodiments, the one or more terminal moieties reduce mRNA expression of a second transcript from other than the target gene.

In certain embodiments, the one or more terminal moieties comprise a 17-mer phosphorothioate backbone DNA oligonucleotide conjugated to the RNAi core at the 5′ end of the sense strand, and targets the second transcript.

In certain embodiments, the one or more terminal moieties prolong the circulation time of the RNAi construct and/or increase the half-life of the RNAi construct in an organism.

In certain embodiments, the one or more terminal moieties promote endocytosis of the RNAi construct into a cell.

In certain embodiments, the one or more terminal moieties are membrane intercalators, or bind to receptors which are internalized into the cell.

In certain embodiments, the one or more terminal moieties carry a charge.

In certain embodiments, the one or more terminal moieties facilitate active or passive transport, localization, or compartmentalization of the RNAi construct.

In certain embodiments, the RNAi core is associated with cellular factors that affect gene expression or are involved in RNA modifications.

In certain embodiments, the terminal moieties are attached directly or via the linker to the RNAi core at a nucleobase position, a sugar position, or one of the terminal internucleoside linkages.

In certain embodiments, the terminal moieties are attached directly or via the linker to the RNAi core at one or both termini or to internal residues.

In certain embodiments, the terminal moieties are attached directly or via the linker to the RNAi core at heterocyclic base moieties (e.g., purines and pyrimidines), monomeric subunits (e.g., sugar moieties), or monomeric subunit linkages (e.g., phosphodiester linkages) of the nucleotides of the RNAi core.

In certain embodiments, the terminal moieties are attached directly or via the linkers to the RNAi core at each end of the sense strand or at each end of the antisense strand.

In certain embodiments, the linkers comprise a chain structure or an oligomer of repeating units.

In certain embodiments, the repeating units are ethylene glyol or amino acid units.

In certain embodiments, the linkers comprise one or more functionalities selected from: an amino group, a hydroxyl group, a carboxylic acid, a thiol group, a phosphoramidate, a phosphate, a phosphite, or an unsaturation (e.g., a double or triple bond).

In certain embodiments, the linkers comprise a nucleic acid hairpin that links the 5′ end of one strand to the 3′ end of the other strand of the dsRNA.

In certain embodiments, the terminal moieties are attached to, incorporated into, or branched from the linkers.

In certain embodiments, the linkers non-covalently bind the RNAi core to the terminal moiety.

In certain embodiments, the linkers are labile.

In certain embodiments, the linkers comprise a divalent group selected from alkylene, cycloalkylene, arylene, heterocyclyl, heteroarylene, and variables thereof.

In certain embodiments, the RNAi construct further comprises a sequestration vehicle that carries, conveys, or holds inactive the RNAi construct.

In certain embodiments, the RNAi constructed is activated by an amount of energy applied to the sequestration vehicle.

In certain embodiments, the sequestration vehicle comprises a liposome, a nanotransporter, a composite, a metal complex or aggregate, a polymer or biopolymer or biocomposite, a nanoparticle, or a microparticle.

In certain embodiments, the polymer comprises one repeating monomer unit, block polymers or co-polymers.

In certain embodiments, the polymer is multimerized, magnetized, charged, neutral, or in the form of micelles.

In certain embodiments, the nanoparticle is an iNOP, an apolipoprotein A-I nanoparticle, a magnetic nanoparticle, MPG peptide nanoparticle, or a quantum dot nanoparticle.

In certain embodiments, the sequestration vehicle comprises two or more layers of biomolecular fabric.

In certain embodiments, the sequestration vehicle is modified for targeting.

In certain embodiments, the sequestration vehicle is modified on the surface or integral to the sequestration vehicle.

In certain embodiments, the RNAi constructed is activated by the energy in either a spatial and/or temporal manner.

In certain embodiments, the energy comprises one or more types of forms.

In certain embodiments, the energy comprises radiant energy (e.g., light, fluorescent, bioluminescence, radiation), ultrasound energy, electricity (charge, varying voltage, electromagnetic), heat (thermal), mechanical energy (pressure), ionic or charge energy, energy held in biologically activated carriers, nuclear energy (fusion and fission), an energy with a wave length between infra red (heat) and x-ray, ultraviolet energy, piezoelectric potential, and/or chemical energy.

In certain embodiments, the sequestration vehicle has a modification on the 5′ end of the anti-sense strand, wherein the modification degrades in the presence of light with a particular wavelength.

In certain embodiments, the sequestration vehicle can be activated locally in an organism systemically administered with the RNAi construct.

In certain embodiments, the sequestration vehicle is activated by an esterase, a phosphatase, or a lipase.

Another aspect of the invention provides a pharmaceutical composition comprising a subject RNAi construct, and one or more pharmaceutically acceptable carriers, excipients, diluents, penetration enhancers, surfactants, and other active or inactive ingredients.

In certain embodiments, the other active ingredients comprise one or more: chemotherapeutic agents which function by a non-RNAi mechanism, or anti-inflammatory drugs.

In certain embodiments, the composition is formulated for oral, topical, pulmonary, inhalation, intratracheal, intranasal, epidermal, transdermal, or parenteral delivery.

Another aspect of the invention provides a pharmaceutical composition for injectable delivery of the subject RNAi construct, and pharmaceutically acceptable carriers, excipients, diluents, penetration enhancers, surfactants, and other active or inactive ingredients for injection.

Another aspect of the invention provides a method of modulating the expression of a target gene in a cell, tissue, or organism, comprising contacting the cell, tissue, or organism with the subject RNAi construct, wherein the antisense strand is at least partially complementary to and hybridizes with a transcript from the target gene.

In certain embodiments, the cell or tissue is contacted with the RNAi construct in vitro or ex vivo.

In certain embodiments, the cell, tissue, or organism is contacted with the RNAi construct in vivo.

In certain embodiments, overexpression of the target gene leads to a disease condition selected from: cancer, retinopathy, autoimmune disease, inflammatory disease, viral disease, miRNA disorder, or cardiovascular disease.

In certain embodiments, the RNAi construct comprises a sequestration vehicle, and the method further comprising: activating the RNAi construct via the application of energy from an energy source sufficient to trigger the release or activation of the RNAi construct from the sequestration vehicle.

In certain embodiments, the method further comprises: measuring the expression level of the target gene in the cell, tissue, or organism.

It is contemplated that any of the embodiments described herein, including those described under different aspects of the invention, can be combined with any other embodiments whenever applicable.

### A

### DETAILED DESCRIPTION OF THE INVENTION

Recently, it has been found that chemically synthesized RNA duplexes of 25-30 base length can have as much as a 100-fold increase in potency of inhibition as compared with 21-mers targeting the same location. In one study of the gene, EGFPS1, only minor differences in potency were seen between duplexes with blunt, 3′-overhang or 5′-overhang ends. A blunt 27-mer duplex was, in fact, most potent (Kim et al., Nat Biotechnol 23: 222-226, 2005). Increased potency has similarly been described for 29-mer stem short hairpin RNAs (shRNAs) when compared with 19-mer stem hairpins (Siolas et al., Nat. Biotechnol. 23: 227-231, 2005).

Certain blunt-ended RNAi constructs were described as early as 2002 in U.S. patent application Ser. Nos. 10/357,529, 10/357,826, and 11/049,636; while pre-annealed double-stranded RNA constructs of 25 base pairs having proprietary modifications in the sense strand are commercially available from Invitrogen Corporation (Carlsbad, Calif.) and are referred to as STEALTH™ RNAi.

### Tripartite RNAi Constructs

The present invention provides further improvements regarding the efficacy of blunt-ended dsRNA constructs for gene silencing. In doing so, one aspect of the present invention is directed to tripartite RNAi constructs which comprise: (1) an RNAi core (either native or abbreviated in form), (2) one or more terminal moieties attached to the RNAi core, and, optionally, (3) a linker between the RNAi core and the terminal moiety. As used herein, the terms “comprising,” “including,” and “containing” are used herein in their open, non-limited sense.

### The RNAi Core

According to the present invention, the “RNAi core” of the constructs or molecules comprises a blunt-ended double-stranded RNA (dsRNA) molecule. RNAi cores may be chemically modified. In one embodiment, modification of the RNAi cores involves the incorporation of 2′ substituent groups at either or both of the 5′ and 3′ ends of the sense strand. More preferably the modification comprises four such modifications in each terminus of the sense strand. In one embodiment, between three and five 2′-O-methyl groups at each of the 5′ and 3′ ends of the sense strand of the dsRNA are incorporated.

In one embodiment, the RNAi core is abbreviated. The abbreviated RNAi core, while based on the native RNAi core in that it retains a contiguous portion of a native core, is configured such that a portion of the native core is substituted with a non-nucleic acid based functional moiety. The effect of the substitution is to reduce the overall size of the dsRNA portion of the construct and to impart to the construct a beneficial feature or property or characteristic. For example, if the native RNAi core (blunt ended dsRNA) is 25 nucleotides in length, then in one embodiment, about 15-20 contiguous nucleotides of the native RNAi core is retained while the remaining 5-10 nucleotides are replaced or substituted with the non-nucleic acid functional moiety (e.g., protein, peptide, carbohydrate, lipid, fat, or any other non-nucleic acid based molecule). Alternatively, the abbreviated RNAi core retains only 7-10 contiguous nucleotide base pairs from the native RNAi core. Constructs containing such an abbreviated RNAi core find uses in applications to modulate gene expression via microRNA (miRNA) pathways.

In one embodiment, the contiguous portion of the native RNAi core is substituted with a peptide or protein structural element such as a binding domain or region. Such elements include, for example, domains, regions or small peptides which are integral to or associated with the RNAi machinery or mechanism.

In one embodiment, the non-nucleic acid functional moiety is a segment, region, domain or portion of a RISC(RNAi Induced Silencing Complex) protein, a RISC complex comprising the RISC protein, or a protein associated with the RISC complex. In this manner, it is believed that incorporation of a binding domain known to interact with the RISC complex improves the affinity of the RNAi construct to the RISC complex, and thus will improve the efficacy of the RNAi construct containing the abbreviated core and binding domain.

In another embodiment of the invention, the abbreviated RNAi core of the invention is associated with a RISC protein component which may further associate with the translation machinery of a cell. Such interaction with the translation machinery of the cell can include interaction with structural and enzymatic proteins of the translation machinery including, but not limited to, the polyribosome and ribosomal subunits.

As used herein the term “double stranded” refers to a construct having two strands. A “double stranded RNA molecule” is one having two strands of RNA which are complementary and hybridizable to one another. It is understood that a double-stranded RNA molecule may be chemically modified and still be considered a double stranded RNA molecule. For sake of convention, the double-stranded RNA molecules of the present invention have a sense strand and an antisense strand. As used herein the term “antisense strand” is that strand of the double stranded molecule which is complementary to at least a portion of an mRNA or RNA species in a cell, tissue or organism while also being complementary to the sense strand of the double stranded molecule of which it is a part. The RNA species being targeted may comprise protein coding mRNA sequence or RNA sequences that do not encode proteins. As used herein the term “sense strand” is that strand of the double stranded molecule which is complementary to the antisense strand.

As used herein “blunt-ends” or “blunt-ended,” as that term applies to double stranded nucleic acid based constructs or molecules, means that the construct or molecule has symmetric termini or termini having no overhanging nucleotides. The two strands of a double stranded molecule align with each other, by virtue of having the same number of nucleotides in each strand, without overhanging nucleotides at the termini. For example, a blunt-ended RNAi molecule comprises terminal nucleotides that are complimentary between the sense and antisense regions of the RNAi molecule.

As used herein the terms “molecule” and “compound” are interchangeable and simply refer to the entity of interest.

In the native form the RNAi cores of the present invention comprise a blunt-ended dsRNA where each strand is between 25-30, preferably 25-27 and more preferably 25 nucleotides in length. The RNAi core molecules of the constructs of the invention which are about 25 to about 30 nucleotides in length are for example about 25-30, 25-27, 25, 26, 27, 27, 29 or 30 nucleotides in length.

In the abbreviated form the RNAi cores of the present invention comprise a blunt-ended dsRNA where each strand is between 7-10, preferably 7-8 and more preferably 8 nucleotides in length. The abbreviated RNAi core molecules of the constructs of the invention which are 25 to about 30 nucleotides in length are for example about 7-10, 7-8, 7, 8, 9, or 10 nucleotides in length.

The term “oligonucleotide,” as used herein, refers to an oligomer or polymer of ribonucleic acid (RNA) or deoxyribonucleic acid (DNA) composed of naturally occurring nucleobases, sugars and phosphodiester internucleoside linkages while the term “nucleotide” refers to the monomer unit of an oligonucleotide. Nucleotides include purines, e.g., adenine, hypoxanthine, guanine, and their derivatives and analogs, as well as pyrimidines, e.g., cytosine, uracil, thymine, and their derivatives and analogs. The term “polynucleotide,” as the name suggests, refers to a polymer of nucleotides and hence is equivalent to an oligonucleotide.

The terms “oligomer” and “oligomeric compound,” as used herein, refer to a plurality of naturally occurring and/or non-naturally occurring nucleosides, joined together with internucleoside linking groups in a specific sequence. At least some of the oligomeric compounds can be capable of hybridizing a region of a target nucleic acid. Included in the terms “oligomer” and “oligomeric compound” are oligonucleotides, oligonucleotide analogs, oligonucleotide mimetics, oligonucleosides and chimeric combinations of these. As such the term oligomeric compound is broader than the term “oligonucleotide,” including all oligomers having all manner of modifications including but not limited to those known in the art. Oligomeric compounds are typically structurally distinguishable from, yet functionally interchangeable with, naturally-occurring or synthetic wild-type oligonucleotides. Thus, oligomeric compounds include all such structures that function effectively to mimic the structure and/or function of a desired RNA or DNA strand, for example, by hybridizing to a target. Such non-naturally occurring oligonucleotides are often desired over the naturally occurring forms because they often have enhanced properties, such as for example, enhanced cellular uptake, enhanced affinity for nucleic acid target and increased stability in the presence of nucleases.

Double-stranded oligomeric compounds include compositions comprising double-stranded constructs such as, for example, two oligomeric compounds forming a double stranded hybridized construct or a single strand with sufficient self complementarity to allow for hybridization and formation of a fully or partially double-stranded compound.

In one embodiment of the invention, double-stranded oligomeric compounds encompass RNAi core molecules.

The term “complementary” refers to the ability of a polynucleotide or oligonucleotide to form base pairs with another polynucleotide or oligonucleotide, or in the case of self-complementarity, within the same polynucleotide or oligonucleotide. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 25-mers, if only five base pairs on each strand can hydrogen bond with each other, the polynucleotide strands exhibit 20% complementarity. In the same example, if 20 base pairs on each strand can hydrogen bond with each other, the polynucleotide strands exhibit 80% complementarity.

As used herein, the phrase “gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi), a pathway whereby RNAi molecules or constructs act, often in concert with host proteins (e.g. the RNA induced silencing complex, RISC) to degrade messenger RNA (mRNA) in a sequence-dependent fashion.

The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g. fluorescent properties (e.g. GFP) or enzymatic activity (e.g. alkaline phosphatases), or several other procedures.

### Chemical Modifications

The RNAi cores of the constructs of the invention may comprise certain modifications to at least one of the two strands from which they are made.

### Bases and Modifications

Naturally occurring bases include, for example, adenine, guanine, cytosine, thymine, uracil, and inosine. While it is preferred that the RNAi cores of the present invention comprise naturally occurring bases, these bases may be modified and incorporated into the RNAi cores of the present invention. Modification may be by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

### Nucleotide Modifications

According to the present invention, at least two of the nucleotides (a base-sugar combination) in the sense strand may comprise modified nucleotides selected from the group consisting of 2′ modified nucleotides. In a preferred embodiment, the modified RNAi core molecule comprises 2′-O-methyl nucleotides (e.g., 2′-O-methyl purine and/or pyrimidine nucleotides) such as, for example, 2′-O-methyl guanosine, 2′-O-methyl uridine nucleotides, 2′-O-methyl adenosine nucleotides, 2′-O-methyl cytosine nucleotides, and mixtures thereof in the sense strand. In a particularly preferred embodiment, the sense strand of the RNAi cores comprise four, 2′-O-methyl modifications at each end of the strand.

Other 2′ modifications which may be incorporated into the RNAi core of the constructs of the invention include OH; F; O-, S-, or N-alkyl; O-, S-, or N-alkenyl; O, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Others include O[(CH₂)ₙO]ₘCH₃, O(CH₂)ₙOCH₃, O(CH₂)ₙNH₂, O(CH₂)ₙCH₃, O(CH₂)ONH₂, and O(CH₂)ₙON[(CH₂)ₙCH₃]₂, where n and m are from 1 to about 10. Others comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

Other modifications shown to be effective in antisense molecules include 2′-methoxyethoxy(2′-O—CH₂CH₂OCH₃, also known as 2′-O-(2-methoxyethyl) or 2′-MOE) (Martin et al., Helv. Chim. Acta, 78: 486-504, 1995), i.e., an alkoxyalkoxy group, 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-aminoethoxy-ethyl or 2′-DMAEOE), i.e., 2′-O—CH₂—O—CH₂—N(CH₃)₂.

Other modifications include 2′-methoxy (2′-O—CH₃), 2′-aminopropoxy (2′-OCH₂CH₂CH₂NH₂), 2′-allyl, 2′-O-allyl and 2′-fluoro (2′-F). The 2′-modification may be in the arabino (up) position or ribo (down) position. One 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Strands of the RNAi cores may also have sugar mimetics such as cyclobutyl moieties in place of the pentofuranosyl sugar.

### Nucleotide Analogs, Modifications and Universal Bases

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

In addition to the natural nucleotides, the term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Nucleotides, as that term is known in the art as representing the base and sugar moieties, may also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

Further, the term nucleotide also includes those species that have a detectable label, such as for example a radioactive or fluorescent moiety, or mass label attached to the nucleotide.

### Internucleoside/Internucleotide (Backbone) Linkages

While native phosphodiester backbone linkages in the RNAi cores are preferred, other backbone linkages may be incorporated into the RNAi cores. Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage, i.e. a single inverted nucleoside residue which may be abasic (the nucleobase is missing or has a hydroxyl group in place thereof). Various salts, mixed salts and free acid forms are also included.

Modified backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Modifications to the Terminal Moieties and Optional Linkers

Attached to the RNAi cores of the present invention are terminal moieties and optionally a linker joining the RNAi core to the terminal moiety. The terminal moiety or the linkers of the present invention may comprise, or be modified by, alkyl, alkenyl, alkynyl, aliphatic, alkoxy, aryl, halo, aromatic, heterocyclic groups or any combination thereof and further any additional protecting or substituent groups.

The term “alkyl,” as used herein, refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms with from 1 to about 6 carbon atoms are also suitable. Alkyl groups as used herein may optionally include one or more further substituent groups.

The term “alkenyl,” as used herein, refers to a straight or branched hydrocarbon chain radical containing up to twenty four carbon atoms having at least one carbon-carbon double bond. Examples of alkenyl groups include, but are not limited to, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms are also suitable. Alkenyl groups as used herein may optionally include one or more further substituent groups.

The term “alkynyl,” as used herein, refers to a straight or branched hydrocarbon radical containing up to twenty four carbon atoms and having at least one carbon-carbon triple bond. Examples of alkynyl groups include, but are not limited to, ethynyl, 1-propynyl, 1-butynyl, and the like. Alkynyl groups typically include from 2 to about 24 carbon atoms, more typically from 2 to about 12 carbon atoms with from 2 to about 6 carbon atoms are also suitable. Alkynyl groups as used herein may optionally include one or more further substituent groups.

The term “aliphatic,” as used herein, refers to a straight or branched hydrocarbon radical containing up to twenty four carbon atoms wherein the saturation between any two carbon atoms is a single, double or triple bond. An aliphatic group can contain from 1 to about 24 carbon atoms, more typically from 1 to about 12 carbon atoms with from 1 to about 6 carbon atoms being desired. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines, for example. Aliphatic groups as used herein may optionally include further substituent groups.

The term “alkoxy,” as used herein, refers to a radical formed between an alkyl group and an oxygen atom wherein the oxygen atom is used to attach the alkoxy group to a parent molecule. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

The terms “halo” and “halogen,” as used herein, refer to an atom selected from fluorine, chlorine, bromine and iodine.

The terms “aryl” and “aromatic,” as used herein, refer to a mono- or polycyclic carbocyclic ring system radical having one or more aromatic rings. Examples of aryl groups include, but not limited to, phenyl, naphthyl, tetrahydronaphthyl, indanyl, idenyl and the like. Aryl groups as used herein may optionally include further substituent groups.

The term “heterocyclic,” as used herein, refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain no heteroatoms. A heterocyclic group typically includes at least one atom selected from sulfur, nitrogen or oxygen. Examples of heterocyclic groups include, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substituent groups.

The terms “substituent and substituent group,” as used herein, are meant to include groups that are typically added to other groups or parent compounds to enhance desired properties or give desired effects. Substituent groups can be protected or unprotected and can be added to one available site or to many available sites in a parent compound, here either the RNAi core or a terminal moiety. When the parent compound is the RNAi core, the terminal moiety can likewise be the substituent group. Substituent groups may also be further substituted with other substituent groups and may be attached directly or via a linking group such as an alkyl or hydrocarbyl group to the parent compound. Such substituent groups include without limitation, halogen, hydroxyl, alkyl, alkenyl, alkynyl, acyl, carboxyl, aliphatic, alicyclic, alkoxy, substituted oxo, aryl, aralkyl, heterocyclic, heteroaryl, heteroarylalkyl, amino, imino, amido, azido, nitro, cyano, carbamido, ureido, thioureido, guanidinyl, amidinyl, thiol, sulfinyl, sulfonyl and sulfonamidyl. Wherein each may comprise a further substituent group which can be, without limitation, alkyl, alkenyl, alkynyl, aliphatic, alkoxy, acyl, aryl, aralkyl, heteroaryl, alicyclic, heterocyclic and heteroarylalkyl.

The term “protecting group,” as used herein, refers to a labile chemical moiety which is known in the art to protect reactive groups including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999).

### Terminal Moieties Attached to the RNAi Core (Directly or Via Linker)

The terminal moieties attached to the RNAi cores of the present invention may be so attached directly or indirectly via a linker. For tripartite RNAi constructs, the linker is present. Functionally, the terminal moieties may be designed to achieve one or more improved outcomes. As used herein the term “terminal moiety” is a compound or molecule or construct which is attached, linked or associated with one or more terminus of the RNAi core. The “terminus of the RNAi core” is further defined to embrace not only the first or final nucleotides of the ends of the dsRNA of the core but also the termini generally. This includes the first 5 nucleotides and the last 5 nucleotides of a native RNAi core. Should the RNAi core be an abbreviated core being 10 or fewer nucleotides in length, the “terminus of the RNAi core” then embraces the first two and last two nucleotides. Where it is intended that the terminal moiety or linker be attached to, and only to, either the first or last nucleotides of either strand of the RNAi core, it will be so noted or claimed. Otherwise, the terms “terminal”, “terminal end” “terminus” carries its usual and customary meaning, modified only as described herein.

In one embodiment, the terminal moieties are designed to act in concert or independently (e.g., when one or more terminal moieties are present on both ends of the RNA core) to protect the RNAi core. In this embodiment, one terminal moiety is attached directly to the core or indirectly via a linker and is long enough or has enough structure such that the terminal moiety protects the RNAi core. Alternatively, terminal moieties may be attached at each end of the RNAi core such that protection is afforded from both ends. Protection of the RNAi core may be afforded by simply enveloping the core or by providing a charged environment such the core is sequestered from the cellular milieu. Any alteration in the presentation or access of the RNAi core to the cellular environment, according to the present invention would constitute a degree of protection. As with chemical modifications, protection means improvements desirable for delivery or efficacy and include increased half-life, reduced degradation, and/or improved delivery profiles, improvement in pharmacokinetic and pharmacodynamic properties and the like.

In one embodiment, the terminal moieties are designed to reduce mRNA expression of an alternate transcript. To this end, the tripartite RNAi construct acts as a bimodal targeting agent. In this embodiment, an antisense, ribozyme or RNAi molecule targeting a gene other than one targeted by the RNAi core-containing construct to which it is attached becomes the “bimodal partner” of the RNAi core. For example a 17-mer phosphorothioate backbone DNA oligonucleotide may be conjugated to the RNAi core (e.g., at the 5′ end of the sense strand) and as such may be designed to target another transcript. This bimodal targeting agent would operate to reduce gene expression in both the nucleus and the cytoplasm. It is understood that the bimodal partner may range from 7-30 nucleobases in length, preferably 15-25. It is understood that the terminal moiety may be 7, 8, 9, 10, 11, 12, 13, 14, 15, 16, 17, 18, 19, 20, 21, 22, 23, 24, 25, 26, 27, 28, 29 or 30 nucleobases in length.

In one embodiment, the terminal moieties attached to the RNAi core prolong the circulation time of the RNAi construct and/or to increase the half-life of the RNAi construct in the organism. Conjugates or terminal moieties known to increase half life include those that facilitate protein binding in the blood and are known to those of skill in the art. It is also known that the lability of the linker may also affect the half life of a construct. Such linkers and their biological stability are known in the art.

In one embodiment the terminal moieties comprise molecules which promote endocytosis of the RNAi construct. As such the terminal moiety acts as a “membrane intercalator.” For example, the membrane intercalators may comprise C₁₀-C₁₈ moieties which may be attached to the 3′ end of antisense strand. These moieties may facilitate or result in the RNAi construct becoming embedded in the lipid bilayer of a cell. Upon “flipping” of the lipids, the RNAi construct would then enter the cell. In these constructs, the linker between the terminal moiety and the RNAi core can be selected such that it is sensitive to the physicochemical environment of the cell and/or to be susceptible to or resistant to enzymes present. The end result being the liberation of the RNAi core, with or without a portion of the optional linker. The present invention also contemplates RNAi constructs that bind to receptors which are internalized.

In one embodiment, the terminal moiety is configured such that it carries a charge. In this embodiment, the terminal moiety may be a nucleic acid or non-nucleic acid polymer which is selected to mimic a carrier (a “carrier mimic”) for the RNAi construct. The terminal moieties in this embodiment are not simply conjugated lipids such as those used in the art (i.e., cationic or anionic lipids). The charge-capped tripartite RNAi constructs may comprise peptide or nucleic acid polymers and be from 10-14 units (amino acids or nucleotides) in length.

Furthermore, the RNAi constructs of the invention itself can have one or more terminal moieties which facilitates the active or passive transport, localization, or compartmentalization of the RNAi construct. Cellular localization includes, but is not limited to, localization to within the nucleus, the nucleolus, or the cytoplasm. Compartmentalization includes, but is not limited to, any directed movement of the oligonucleotides of the invention to a cellular compartment including the nucleus, nucleolus, mitochondrion, or imbedding into a cellular membrane surrounding a compartment or the cell itself.

In another embodiment of the invention, the RNAi core of the invention is associated with cellular factors that affect gene expression, more specifically those involved in RNA modifications. These modifications include, but are not limited to, post-transcriptional modifications such as methylation.

### Conjugates as Terminal Moieties

Terminal moieties, while attached directly to the RNAi core or to the RNAi core via an optional linker may comprise conjugate groups attached to one or more of the RNAi core termini at selected nucleobase positions, sugar positions or to one of the terminal internucleoside linkages.

There are numerous methods for preparing conjugates of RNAi cores. Generally, an RNAi core is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic. For example, an electrophilic group can be a carbonyl-containing functionality and a nucleophilic group can be an amine or thiol. Methods for conjugation of nucleic acids and related compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In some embodiments, conjugate moieties can be attached to the terminus of an RNAi core such as a 5′ or 3′ terminal residue of either strand. Conjugate moieties can also be attached to internal residues of the oligomeric compounds. For RNAi cores, conjugate moieties can be attached to one or both strands. In some embodiments, a double-stranded RNAi core contains a conjugate moiety attached to each end of the sense strand. In other embodiments, a double-stranded RNAi core contains a conjugate moiety attached to both ends of the antisense strand.

In some embodiments, conjugate moieties can be attached to heterocyclic base moieties (e.g., purines and pyrimidines), monomeric subunits (e.g., sugar moieties), or monomeric subunit linkages (e.g., phosphodiester linkages) of nucleic acid molecules. Conjugation to purines or derivatives thereof can occur at any position including, endocyclic and exocyclic atoms. In some embodiments, the 2-, 6-, 7-, or 8-positions of a purine base are attached to a conjugate moiety. Conjugation to pyrimidines or derivatives thereof can also occur at any position. In some embodiments, the 2-, 5-, and 6-positions of a pyrimidine base can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms.

Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

These terminal moieties act to enhance the properties of the RNAi construct or may be used to track the RNAi construct or its metabolites and/or effect the trafficking of the construct. Properties that are typically enhanced include without limitation activity, cellular distribution and cellular uptake. In one embodiment, the RNAi constructs are prepared by covalently attaching the terminal moieties to chemically functional groups available on the RNAi core or linker such as hydroxyl or amino functional groups. Conjugates which may be used as terminal moities include intercalators, reporter molecules, polyamines, polyamides, polyethylene glycols, polyethers, and groups that enhance the pharmacodynamic and/or pharmacokinetic properties of the RNAi construct.

Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve properties including but not limited to construct uptake, construct resistance to degradation, and/or strengthen sequence-specific hybridization with RNA.

Conjugate groups also include but are not limited to lipid moieties such as a cholesterol moiety, cholic acid, a thioether, an aliphatic chain, a phospholipid, a polyamine or a polyethylene glycol chain or adamantane acetic acid, a palmityl moiety or an octadecylamine or hexylamino-carbonyl-oxycholesterol moiety.

The RNAi cores of the invention may also be conjugated to active drug substances. Representative U.S. patents that teach the preparation of such conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941 each of which are incorporated herein by reference in their entirety.

The present invention provides, inter alia, RNAi constructs and compositions containing the same wherein the terminal moiety comprises one or more conjugate moieties. The terminal moieties (e.g., conjugates) of the present invention can be covalently attached, optionally through one or more linkers, to one or more RNAi cores. The resulting constructs can have modified or enhanced pharmacokinetic, pharmacodynamic, and other properties compared with non-conjugated constructs. A conjugate moiety that can modify or enhance the pharmacokinetic properties of an RNAi construct can improve cellular distribution, bioavailability, metabolism, excretion, permeability, and/or cellular uptake of the RNAi construct. A conjugate moiety that can modify or enhance pharmacodynamic properties of an RNAi construct can improve activity, resistance to degradation, sequence-specific hybridization, uptake, and the like.

Representative conjugate moieties can include lipophilic molecules (aromatic and non-aromatic) including steroid molecules; proteins (e.g., antibodies, enzymes, serum proteins); peptides; vitamins (water-soluble or lipid-soluble); polymers (water-soluble or lipid-soluble); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonuclease/intercalators; crosslinking agents (e.g., photoactive, redox active), and combinations and derivatives thereof. Oligonucleotide conjugates and their syntheses are also reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense & Nucleic Acid Drug Development, 2002, 12, 103, each of which is incorporated herein by reference in its entirety.

Lipophilic conjugate moieties can be used, for example, to counter the hydrophilic nature of an RNAi construct and enhance cellular penetration. Lipophilic moieties include, for example, steroids and related compounds such as cholesterol (U.S. Pat. No. 4,958,013 and Letsinger et al., Proc. Natl. Acad. Sci. USA, 1989, 86, 6553), thiocholesterol (Oberhauser et al., Nuc. Acids Res., 1992, 20, 533), lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, 17-hydroxycorticosterone, their derivatives, and the like.

Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include undecyl, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O, S, or N (e.g., geranyloxyhexyl). Further suitable lipophilic conjugate moieties include aliphatic derivatives of glycerols such as alkylglycerols, bis(alkyl)glycerols, tris(alkyl)glycerols, monoglycerides, diglycerides, and triglycerides. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosanoic acids and the like.

In further embodiments, lipophilic conjugate groups can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like.

Other suitable lipophilic conjugate moieties include menthols, trityls (e.g., dimethoxytrityl (DMT)), phenoxazines, lipoic acid, phospholipids, ethers, thioethers (e.g., hexyl-S-tritylthiol), derivatives thereof and the like. RNAi constructs containing conjugate moieties with affinity for low density lipoprotein (LDL) can help provide an effective targeted delivery system. High expression levels of receptors for LDL on tumor cells makes LDL an attractive carrier for selective delivery of drugs to these cells (Rump et al., Bioconjugate Chem. 9: 341, 1998; Firestone, Bioconjugate Chem. 5: 105, 1994; Mishra et al., Biochim. Biophys. Acta 1264: 229, 1995). Moieties having affinity for LDL include many lipophilic groups such as steroids (e.g., cholesterol), fatty acids, derivatives thereof and combinations thereof. In some embodiments, conjugate moieties having LDL affinity can be dioleyl esters of cholic acids such as chenodeoxycholic acid and lithocholic acid.

Conjugate moieties can also include vitamins. Vitamins are known to be transported into cells by numerous cellular transport systems. Typically, vitamins can be classified as water soluble or lipid soluble. Water soluble vitamins include thiamine, riboflavin, nicotinic acid or niacin, the vitamin B₆ pyridoxal group, pantothenic acid, biotin, folic acid, the B₁₂ cobamide coenzymes, inositol, choline and ascorbic acid. Lipid soluble vitamins include the vitamin A family, vitamin D, the vitamin E tocopherol family and vitamin K (and phytols).

In some embodiments, the conjugate moiety includes folic acid (folate) and/or one or more of its various forms, such as dihydrofolic acid, tetrahydrofolic acid, folinic acid, pteropolyglutamic acid, dihydrofolates, tetrahydrofolates, tetrahydropterins, 1-deaza, 3-deaza, 5-deaza, 8-deaza, 10-deaza, 1,5-dideaza, 5,10-dideaza, 8,10-dideaza and 5,8-dideaza folate analogs, and antifolates.

Vitamin conjugate moieties include, for example, vitamin A (retinol) and/or related compounds. The vitamin A family (retinoids), including retinoic acid and retinol, are typically absorbed and transported to target tissues through their interaction with specific proteins such as cytosol retinol-binding protein type II (CRBP-II), retinol binding protein (RBP), and cellular retinol-binding protein (CRBP). The vitamin A family of compounds can be attached to an RNAi core via acid or alcohol functionalities found in the various family members. For example, conjugation of an N-hydroxy succinimide ester of an acid moiety of retinoic acid to an amine function on a linker pendant to an RNAi core can result in linkage of vitamin A compound to the RNAi core via an amide bond. Also, retinol can be converted to its phosphoramidite, which is useful for 5′ conjugation.

alpha-Tocopherol (vitamin E) and the other tocopherols (beta through zeta) can be conjugated to RNAi cores to enhance uptake because of their lipophilic character. Also, vitamin D, and its ergosterol precursors, can be conjugated to RNAi cores through their hydroxyl groups by first activating the hydroxyl groups to, for example, hemisuccinate esters. Conjugation can then be effected directly to the RNAi core or to an amino linker pendant from the RNAi core. Other vitamins that can be conjugated to RNAi cores in a similar manner on include thiamine, riboflavin, pyridoxine, pyridoxamine, pyridoxal, deoxypyridoxine. Lipid soluble vitamin K's and related quinone-containing compounds can be conjugated via carbonyl groups on the quinone ring. The phytol moiety of vitamin K can also serve to enhance binding of the oligomeric compounds to cells.

Pyridoxal (vitamin B₆) has specific B₆-binding proteins. Other pyridoxal family members include pyridoxine, pyridoxamine, pyridoxal phosphate, and pyridoxic acid. Pyridoxic acid, niacin, pantothenic acid, biotin, folic acid and ascorbic acid can be conjugated to RNAi cores, for example, using N-hydroxysuccinimide esters that are reactive with amino linkers located on the RNAi core, as described above for retinoic acid.

Vitamin conjugate moieties can also be used to facilitate the targeting of specific cells or tissues. For example, vitamin D and analogs thereof can assist in transporting conjugated RNAi cores or constructs to keratinocytes, dermal fibroblasts, and other cells containing vitamin D₃ nuclear receptors. Additionally, Vitamin A and other retinoids can be used to target cells with retinoid X receptors. Accordingly, vitamin-containing conjugate moieties can be useful in treating, for example, skin disorders such as psoriasis.

Conjugate moieties can also include polymers. Polymers can provide added bulk and various functional groups to affect permeation, cellular transport, and localization of the conjugated RNAi core. For example, increased hydrodynamic radius caused by conjugation of an RNAi core with a polymer can help prevent entry into the nucleus and encourage localization in the cytoplasm. In some embodiments, the polymer does not substantially reduce cellular uptake or interfere with hybridization to a complementary strand or other target. In further embodiments, the conjugate polymer moiety has, for example, a molecular weight of less than about 40, less than about 30, or less than about 20 kDa. Additionally, polymer conjugate moieties can be water-soluble and optionally further comprise other conjugate moieties such as peptides, carbohydrates, drugs, reporter groups, or further conjugate moieties.

In some embodiments, polymer conjugates include polyethylene glycol (PEG) and copolymers and derivatives thereof. Conjugation to PEG has been shown to increase nuclease stability of nucleic acid based compounds. PEG conjugate moieties can be of any molecular weight including for example, about 100, about 500, about 1000, about 2000, about 5000, about 10,000 and higher. In some embodiments, the PEG conjugate moieties contains at least 4, at least 5, at least 6, at least 7, at least 8, at least 9, at least 10, at least 15, at least 20, or at least 25 ethylene glycol residues. In further embodiments, the PEG conjugate moiety contains from about 4 to about 10, about 4 to about 8, about 5 to about 7, or about 6 ethylene glycol residues. The PEG conjugate moiety can also be modified such that a terminal hydroxyl is replaced by alkoxy, carboxy, acyl, amido, or other functionality. Other conjugate moieties, such as reporter groups including, for example, biotin or fluorescein can also be attached to a PEG conjugate moiety. Copolymers of PEG are also suitable as conjugate moieties. Preparation and biological activity of polyethylene glycol conjugates of oligonucleotides are described, for example, in Bonora et al., Nucleosides Nucleotides 18: 1723, 1999; Bonora et al., Farmaco 53: 634, 1998; Efimov, Bioorg. Khim. 19: 800, 1993; and Jaschke et al., Nucleic Acids Res. 22: 4810, 1994. Further example PEG conjugate moieties and preparation of corresponding conjugated oligomeric compounds is described in, for example, U.S. Pat. Nos. 4,904,582 and 5,672,662, each of which is incorporated by reference herein in its entirety. Nucleic acid compounds conjugated to one or more PEG moieties are available commercially.

Other polymers suitable as conjugate moieties include polyamines, polypeptides, polymethacrylates (e.g., hydroxylpropyl methacrylate (HPMA)), poly(L-lactide), poly(DL lactide-co-glycolide (PGLA), polyacrylic acids, polyethylenimines (PEI), polyalkylacrylic acids, polyurethanes, polyacrylamides, N-alkylacrylamides, polyspermine (PSP), polyethers, cyclodextrins, derivatives thereof and co-polymers thereof. Many polymers, such as PEG and polyamines have receptors present in certain cells, thereby facilitating cellular uptake. Polyamines and other amine-containing polymers can exist in protonated form at physiological pH, effectively countering an anionic backbone of some oligomeric compounds, effectively enhancing cellular permeation. Some example polyamines include polypeptides (e.g., polylysine, polyomithine, polyhistadine, polyarginine, and copolymers thereof), triethylenetetramine, spermine, polyspermine, spermidine, synnorspermidine, C-branched spermidine, and derivatives thereof. Other amine-containing moieties can also serve as suitable conjugate moieties due to, for example, the formation of cationic species at physiological conditions. Example amine-containing moieties include 3-aminopropyl, 3-(N,N-dimethylamino)propyl, 2-(2-(N,N-dimethylamino)ethoxy)ethyl, 2-N-(2-aminoethyl)-N-methylaminooxy)ethyl, 2-(1-imidazolyl)ethyl, and the like.

Conjugate moieties can also include peptides. Suitable peptides can have from 2 to about 30, 2 to about 20, 2 to about 15, or 2 to about 10 amino acid residues. Amino acid residues can be naturally or non-naturally occurring, including both D and L isomers.

In some embodiments, peptide conjugate moieties are pH sensitive peptides such as fusogenic peptides. Fusogenic peptides can facilitate endosomal release of agents such as RNAi cores to the cytoplasm. It is believed that fusogenic peptides change conformation in acidic pH, effectively destabilizing the endosomal membrane thereby enhancing cytoplasmic delivery of endosomal contents. Example fusogenic peptides include peptides derived from polymyxin B, influenza HA2, GALA, KALA, EALA, melittin-derived peptide, α-helical peptide or Alzheimer β-amyloid peptide, and the like. Preparation and biological activity of oligonucleotides conjugated to fusogenic peptides are described in, for example, Bongartz et al., Nucleic Acids Res. 22: 4681, 1994, and U.S. Pat. Nos. 6,559,279 and 6,344,436.

Other peptides that can serve as conjugate moieties include delivery peptides which have the ability to transport relatively large, polar molecules (including peptides, oligonucleotides, and proteins) across cell membranes. Example delivery peptides include Tat peptide from HIV Tat protein and Ant peptide from Drosophila antenna protein. Conjugation of Tat and Ant with oligonucleotides is described in, for example, Astriab-Fisher et al., Biochem. Pharmacol. 60: 83, 2000.

Conjugated delivery peptides can help control localization of RNAi cores and constructs to specific regions of a cell, including, for example, the cytoplasm, nucleus, nucleolus, and endoplasmic reticulum (ER). Nuclear localization can be effected by conjugation of a nuclear localization signal (NLS). In contrast, cytoplasmic localization can be facilitated by conjugation of a nuclear export signal (NES). Methods for conjugating peptides to oligomeric compounds such as oligonucleotides is described in, for example, U.S. Pat. No. 6,559,279, which is incorporated herein by reference in its entirety.

Many drugs, receptor ligands, toxins, reporter molecules, and other small molecules can serve as conjugate moieties. Small molecule conjugate moieties often have specific interactions with certain receptors or other biomolecules, thereby allowing targeting of conjugated RNAi constructs to specific cells or tissues.

In yet further embodiments, small molecule conjugates can target or bind certain receptors or cells. T-cells are known to have exposed amino groups that can form Schiff base complexes with appropriate molecules. Thus, small molecules containing functional groups such as aldehydes that can interact or react with exposed amino groups can also be suitable conjugate moieties.

Reporter groups that are suitable as conjugate moieties include any moiety that can be detected by, for example, spectroscopic means. Example reporter groups include dyes, fluorophores, phosphors, radiolabels, and the like. In some embodiments, the reporter group is biotin, flourescein, rhodamine, coumarin, or related compounds. Reporter groups can also be attached to other conjugate moieties. In one embodiment, the modification to the RNAi constructs may take the form of the addition of a photon cleavable group. Such groups have been disclosed by Nguyen et al., Biochim. Biophys. Acta 1758(3): 394-403, 2006, and are commercially available as light controllable groups from Panomics, Inc. (Fremont, Calif.).

Other conjugate moieties can include proteins, subunits, or fragments thereof. Proteins include, for example, enzymes, reporter enzymes, antibodies, receptors, and the like. In some embodiments, protein conjugate moieties can be antibodies or fragments. Antibodies can be designed to bind to desired targets such as tumor and other disease-related antigens. In further embodiments, protein conjugate moieties can be serum proteins. In yet further embodiments, RNAi cores can be conjugated to RNAi-related proteins, RNAi-related protein complexes, subunits, and fragments thereof. For example, oligomeric compounds can be conjugated to Dicer or RISC or fragments thereof. RISC is a ribonucleoprotein complex that contains an oligonucleotide component and proteins of the Argonaute family of proteins, among others. Argonaute proteins make up a highly conserved family whose members have been implicated in RNA interference and the regulation of related phenomena. Members of this family have been shown to possess the canonical PAZ and Piwi domains, thought to be a region of protein-protein interaction. Other proteins containing these domains have been shown to effect target cleavage, including the RNAse, Dicer.

Other conjugate moieties can include, for example, oligosaccharides and carbohydrate clusters; a glycotripeptide that binds to Gal/GalNAc receptors on hepatocytes, lysine-based galactose clusters; and cholane-based galactose clusters (e.g., carbohydrate recognition motif for asialoglycoprotein receptor). Further suitable conjugates can include oligosaccharides that can bind to carbohydrate recognition domains (CRD) found on the asialoglycoprotein-receptor (ASGP-R).

In further embodiments, cleaving agents can serve as conjugate moieties. Cleaving agents can facilitate degradation of target, such as target nucleic acids, by hydrolytic or redox cleavage mechanisms. Cleaving groups that can be suitable as conjugate moieties include, for example, metallocomplexes, peptides, amines, enzymes, and constructs containing constituents of the active sites of nucleases.

Cross-linking agents can also serve as conjugate moieties. Cross-linking agents facilitate the covalent linkage of the conjugated RNAi cores with other compounds. In some embodiments, cross-linking agents can covalently link double-stranded nucleic acids, effectively increasing duplex stability and modulating pharmacokinetic properties. In some embodiments, cross-linking agents can be photoactive or redox active. Other suitable conjugate moieties include, for example, polyboranes, carboranes, metallopolyboranes, metallocarborane, derivatives thereof and the like.

### Linkers Joining the RNAi Core and the Terminal Moiety(ies)

The tripartite RNAi constructs of the present invention preferably comprise a tether, linker or other group distinct from and positioned between the RNAi core and the terminal moiety. As described above, terminal moieties (conjugates) can be attached to the RNAi core directly or through a linking moiety (linker or tether). Linkers are bifunctional moieties that serve to covalently connect a conjugate moiety to an RNAi core. In some embodiments, the linker comprises a chain structure or an oligomer of repeating units such as ethylene glycol or amino acid units. The linker can have at least two functionalities, one for attaching to the RNAi core and the other for attaching to the terminal (e.g., conjugate) moiety. Example linker functionalities can be electrophilic for reacting with nucleophilic groups on the RNAi core or terminal moiety, or nucleophilic for reacting with electrophilic groups. In some embodiments, linker functionalities include amino, hydroxyl, carboxylic acid, thiol, phosphoramidate, phosphate, phosphite, unsaturations (e.g., double or triple bonds), and the like.

A wide variety of linker groups are known in the art that can be useful in the attachment of terminal moieties to RNAi cores. A review of many of the useful linker groups can be found in, for example, Antisense Research and Applications, S. T. Crooke and B. Lebleu, Eds., CRC Press, Boca Raton, Fla., 1993, p. 303-350. Any of the reported groups can be used as a single linker or in combination with one or more further linkers.

Linkers and their use in preparation of conjugates of oligonucleotides are provided throughout the art. For example, see U.S. Pat. Nos. 4,948,882; 5,525,465; 5,541,313; 5,545,730; 5,552,538; 5,580,731; 5,486,603; 5,608,046; 4,587,044; 4,667,025; 5,254,469; 5,245,022; 5,112,963; 5,391,723; 5,510,475; 5,512,667; 5,574,142; 5,684,142; 5,770,716; 6,096,875; 6,335,432; and 6,335,437, each of which is incorporated by reference in its entirety.

In one embodiment, the linker may comprise a nucleic acid hairpin which links the 5′ end of one strand to the 3′ end of the other strand of the dsRNA RNAi core. When formed from only one strand, the dsRNA RNAi core takes the form of a self-complementary hairpin-type molecule that doubles back on itself to form an RNA duplex. This leaves the opposite terminus of the RNAi core available for modification with a terminal moiety. Alternatively, in this configuration the terminal moiety may be attached to, incorporated into or branched from the linker (nucleic acid hairpin) itself, thus placing the components of the tripartite structure in the preferred orientation of RNAi core-linker-terminal moiety.

Alternatively, the two strands can be linked via a non-nucleic acid linker. Thus, the dsRNAs can be fully or partially double-stranded. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

The term “linking moiety,” or “linker” as used herein is generally a bi-functional group, molecule or compound. It may covalently or non-covalently bind the RNAi core to the terminal moiety. The covalent binding may be at both or only one end of the linker. Whether the nature of binding to the RNAi core and terminal moiety is either covalent or noncovalent, the linker itself may be labile. As used herein the term “labile” as it applies to linkers means that the linker is either temporally or spatially stable for only a definite period or under certain environmental conditions. For example, a labile linker may lose integrity at a certain, time, temperature, pH, pressure, or under a certain magnetic field or electric field. The result of lost integrity being the severance of the connection between the RNAi core and one or more terminal moieties.

Suitable linking moieties or linkers include, but are not limited to, divalent group such as alkylene, cycloalkylene, arylene, heterocyclyl, heteroarylene, and the other variables are as described herein.

### Sequestration Vehicles

Another embodiment of the invention relates to the maintenance of the activity of molecules which function via the RNAi mechanism after delivery to a cell or organism, which has been a focus of much investigation. The present invention provides methods and compositions for the spatial and temporal control over the activity of RNAi molecules via sequestration followed by energy activation.

According to the present invention, RNAi constructs (such as the tripartite RNAi constructs disclosed herein) or portions thereof (such as the RNAi cores disclosed herein) are sequestered such that they remain inactive until such time as a certain amount and/or type of energy is applied to the system containing the construct. Upon energy activation, the RNAi constructs or RNAi cores are released from a sequestered state. As used herein “sequestration” or “being sequestered” or the “sequestered state” is defined as the separating or segregating of an RNAi construct or RNAi core of the present invention such that it is not available for reactions. Sequestration includes the states of being caged, locked, encapsulated, compacted, compressed, contained, centralized, concentrated, congealed, coalesced, gelled, arranged, incorporated, united, conjoined, connected, pooled, secured, stabilized or otherwise maintained in any way in inactive form until such time that the appropriate type and quanta of energy is applied to the system containing the sequestered RNAi construct or RNAi core.

A “sequestration vehicle” as used in the present invention is one in which the RNAi constructs or RNAi cores of the present invention are carried, conveyed or held inactive until such time as the appropriate type and quanta (amount) of energy is applied to the system containing the sequestered RNAi construct or RNAi core.

Sequestration vehicles of the present invention include, but are not limited to, liposomes, nanotransporters, composites, metal complexes or aggregates, polymers or biopolymers or biocomposites such as hydroxyapatite, nanoparticles, microparticles or any other vehicle considered useful for the targeted delivery of nucleic acid constructs. Polymeric sequestration vehicles may be comprised of one repeating monomer unit, block polymers or co-polymers. They may also be multimerized, magnetized, charged, neutral or in the form of micelles. Each of these types of sequestration vehicles, their manufacture and use in the field of nucleic acid research are known in the art.

In one embodiment of the invention the sequestration vehicles are nanoparticles, for example the iNOPs such as those described by Baigude et al., ACS Chem. Biol. 2 (4): 237-241, 2007. The nanoparticle system chosen as the sequestration vehicle may also comprise the apolipoprotein A-I nanoparticle systems (Kim et al., Mol Ther. 15(6): 1145-1152, 2007); magnetic nanoparticles (Medarova et al., Nat Med. 13(3): 372-377, 2000); MPG peptide nanoparticles (Crombez et al., Biochem. Soc. Trans. 35(Pt 1): 44-46, 2007); or quantum dot nanoparticles (Tan et al., Biomaterials 28(8): 1565-1571, 2007).

In one embodiment, the sequestration vehicles are liposomes or lipid-based vehicles as are known in the art and described herein.

In one embodiment, the sequestration vehicle comprises two or more layers of biomolecular fabric. As used herein, a “biomolecular fabric” is a polymer or composite which is biocompatible or biological in origin which can be formed into sheet like or laminar structures. Such biomolecular fabrics include hydroxyapatite, sheets of membrane lipids, cytoskeletal protein webs or meshes, and the like.

The sequestration vehicle may be modified for targeting where a modification is on the surface or integral to the sequestration vehicle. Surface modification of an LPD (liposome-polycation-DNA) nanoparticle system has been disclosed for tumor targeting with canonical siRNA (Li and Huang, Ann. N Y. Acad. Sci. 1082: 1-8, 2006). Modification to the sequestration vehicles may be made in any manner as those made to the RNAi construct or RNAi cores as described below, such as those conjugates which may be appended.

One of skill in the art will appreciate the application and methods of incorporation of nucleic acid based constructs such as the RNAi constructs and RNAi cores of the present invention into sequestration vehicles known in the art without any undue experimentation. Consequently, it should be understood that a novel feature of the invention lies in the energy activation of sequestration vehicles comprising RNAi constructs or RNAi cores of the present invention.

### Energy Activation

For certain therapeutic applications, it would be beneficial to hold the RNAi constructs or the RNAi cores in a stable but inactive state until such time that they reach the target site, tissue or organ. According to the present invention, this is accomplished by sequestration of the RNAi construct or RNAi core followed by energy activation in either a spatial and/or temporal manner. Energy activation may be by one or more types of energy and at one or more time points.

As used herein, an RNAi construct or RNAi core in an “inactive form” is one that would not function to elicit or alter gene expression (e.g., upregulation or down regulation) via an RNAi mechanism. Inactivity may result from sequestration or from modification prior to sequestration. Regarding the activity of the RNAi constructs or RNAi cores, while not wishing to be bound by any particular theory, the inventors contemplate that it may be simply the sequestration of the RNAi constructs and/or RNAi cores that is responsible for the inactivity. For example, RNAi cores contained within the sequestration vehicle may be effective to alter gene expression but for their being sequestered and unable to come into contact with the RNAi machinery of the cell. These constructs would obviously be active prior to incorporation into the sequestration vehicle and hence active RNAi agents. Alternatively, the RNAi constructs or RNAi cores may only become active once released from the sequestration vehicle upon energy activation. For example, chemical modifications may be made to RNAi cores or constructs such that they would not function in the RNAi pathway until released from the sequestration vehicle. As used herein, “energy activation” or “activation by application of energy” is the process of supplying a type and amount of energy sufficient to release the RNAi construct or RNAi core from the sequestration vehicle and hence their inactive state, the release being synonymous with activation of the RNAi construct or RNAi core.

As used herein the term “energy” means the capacity to do work.

Forms and/or sources of energy include wave, radiant (light, fluorescent, bioluminescence, radiation), ultrasound, electricity (charge, varying voltage, electromagnetic), heat (thermal), mechanical (pressure), potential energy held in biologically activated carriers, nuclear energy (fusion and fission), an energy with a wave length between infra red (heat) and x-ray, ultraviolet energy, piezoelectric potential, and chemical energy.

Light activation is also embraced by the present invention (e.g. attachment of a light sensitive protecting group on the end of an siRNA that is removed or leaves when exposed to a certain wave length).

In one embodiment, RNAi constructs have modification(s) which will degrade in the presence of light. The modification can be used as an activator of the RNAi construct activity by being placed on the 5′ end of the anti-sense strand. This will render the molecule inactive until light at a particular wavelength is exposed to the molecule and degrades or alters the chemical modification on the RNAi construct so that it will no longer hinder its ability to silence the target gene. Additionally, light activation of RNAi constructs will allow a systemic delivery approach to work while being able to only activate RNAi molecules in localized, desired regions of therapy.

Activation of these molecules could be accomplished through an external light source for sub-dermal applications (e.g. apply RNAi to a localized area and then expose the outer dermal area to light at a certain wavelength and intensity to activate the constructs). Localized activation of RNAi constructs could be achieved internally by delivering RNAi systemically and then through the use of a small, surgically applied device (e.g. fiber optics, light on camera) that can deliver light to activate the constructs. Applications where this would be beneficial are where you want to activate RNAi constructs which are harmful for normal cells but beneficial in destroying cancerous and/or diseased cells in localized internal regions. Examples of this could be using RNAi to prevent tumor growth/re-growth. Typically tumors in localized internal regions are removed and then patients are subjected to whole body therapies such as chemotherapy and/or radiation. Before surgery a patient could be administered systemically (e.g., intravenous) RNAi constructs that can only be activated by light at a certain wavelength. After the tumor is removed the affected region could be subjected to light to activate RNAi constructs that promote cell death. The region where the RNAi is activated is small and localized to where the tumor was and potentially other cancerous cell may still be. This type of therapy would eliminate a whole body treatment (radiation, chemotherapy) that is typically used to prevent cancer cells from re-growing tumors. Utilizing light allows a minimally invasive procedure to insert a device that will act as the light source to activate the RNAi constructs.

It is also expected that the activity of esterases, phosphatases or lipases in vivo that cleave of a group to free up the active siRNA may be exploited.

### Heat (Thermal) Energy Activation

In one embodiment, energy activation results from the application of heat (thermal) energy to a system containing a sequestration vehicle comprising the RNAi constructs or RNAi cores of the present invention. The application of heat for energy activation, while most often provided by an external energy source, need not come from a source external to the cell, tissue or organism. The heat (thermal) energy may be provided by the cell, tissue or organism itself. For example, upon delivery or administration of a sequestration vehicle comprising an RNAi construct or RNAi core which is held inactive therein or thereon, the increase in available heat which contacts the sequestration vehicle, over that experienced in the ambient atmosphere, outside the organism may be sufficient for activation. Alternatively, the present invention may advantageously take advantage of heat fluctuations of the organism such as those occurring or associated with the circadian rhythm, menstrual cycle (especially the increase in body temperature which occurs within 24 hours of ovulation), during medical treatments (such as treatments for cancer which cause a rise in body temperature), fever, exercise, and/or hypo- or hyperthermia.

In one embodiment, therapeutic compositions comprising RNAi constructs or RNAi cores contained in sequestration vehicles designed to be delivered in a daily dosing regimen may be applied locally (e.g., topically such as with creams or patches), via inhalation, or internally (e.g., systemic administration or by implantation) and then released upon a rise in body temperature via either the breaking of a temperature sensitive or heat labile bond between the RNAi construct or core and the sequestration vehicle, or by the thermal melting of the sequestration vehicle to release the therapeutic compound.

Furthermore, the heat energy provided by the organism and necessary for activation need not be of a level immediately sufficient for activation. For example, sequestration vehicles which have been chemically modified to target a particular organ may advantageously be designed for activation on reaching a warmer or cooler targeted organ.

In this embodiment, heat stability and lability are exploited. Where RNAi constructs or RNAi cores are contained within or encapsulated by a sequestration vehicle, the sequestration vehicle is chosen such that their walls are compromised, degraded or melt at a particular temperature such that the RNAi constructs or cores contained therein are released (i.e., activated). Temperature sensitive polymers are disclosed for example, in U.S. Pat. No. 5,053,228. Where the RNAi constructs or RNAi cores are bound to the sequestration vehicle, such as via chemical bonds or linking groups, then the connection or linkage between the RNAi construct or RNAi core and the sequestration vehicle is chosen such that it will break at a particular temperature, releasing the RNAi construct or core.

### Mechanical Activation

In one embodiment, RNAi constructs or RNAi cores are formulated in liposomal sequestration vehicles. These find uses in topical application such as lotions and creams where the energy activation is accomplished by the mere pressure and/or friction necessary to apply the lotion or cream to the target area.

### Piezoelectric Activation

Sequestration of RNAi constructs or RNAi cores in biomolecular fabrics are advantageous in that they can be assembled by depositing the constructs or cores onto or between layers or by impregnating or embedding or infusing the RNAi constructs or RNAi cores into the composite. For example, hydroxyapatite, a component of bone may be formed in layers with the RNAi constructs and cores deposited between layers. It may further be formed into thicker layers with the RNAi constructs and RNAi cores diffused into the pores of the hydroxyapatite. This composition could then be used in implants or in any application where the absorption of the hydroxyapatite by the cell, tissue or organism (chemical metabolism of the composite representing the energy applied) would occur. The rate of release (activation) of the RNAi constructs or cores upon dissolution of the hydroxyapatite could be predetermined and controlled by controlling the formation or crystallization, hence the porosity, of the composite. Alternatively, since bone and bone matrices respond to piezoelectric effects, a sequestration vehicle comprising hydroxyapatite as the biomolecular fabric could also be designed to release the RNAi construct or core in response to pressure and the concomitant electric charge produced thereby.

### Ionic or Charge Activation

In one embodiment, the sequestration vehicles comprise an RNAi construct or RNAi core in addition to an ionic species. Ionic species include ions and small compounds which may carry a positive or negative charge. These may include H⁺ ions (e.g., as related to pH; for example late endosomes have low pH, which might trigger the release/activation of RNAi core).

The include biological signaling molecules, second messengers, ions such as calcium, magnesium, sodium, manganese, phosphate, nitric oxide, potassium, chloride, biological salts, and the like.

Such ionic species when released from the sequestration vehicle may affect cellular events including, but not limited to, induction of a charged state, neutralization of a charge state, creation or elimination of a charge gradient or triggering of an ion channel, any of which may in turn affect the efficacy or potency of the now activated RNAi construct or RNAi core. It will be appreciated that the ionic species if associated with the exterior or surface of the sequestration vehicle may act to provide the activation energy for the RNAi construct or core. For example, an ion (ionic species) which triggers a voltage gated ion channel may be conjugated or complexed to the surface of the sequestration vehicle. Upon contact with a cell having receptors for the ion or the ion channel, the sequestration vehicle with its complexed or associated ionic species would trigger not only the ion channel or receptor but the release of the RNAi construct or core in one or more ways. In one manner, if the ionic species complexed to the sequestration vehicle was acting a stabilizing agent, then stripping away of the ionic species would result in destabilization of the sequestration vehicle and activation of the RNAi construct or core.

### Biologically Activated Carrier Activation

In addition to the RNAi construct or RNAi core the sequestration vehicle may comprise, contain or be modified with a biologically activated carrier. These activated carriers are evolutionarily specialized to carry high-energy electrons, hydrogen atoms or chemical groups. Biologically activated carriers include, but are not limited to ATP (adenosine triphosphate), NAD (nicotinamide adenine dinucleotide) and the closely related molecule NADP⁺ (nicotinamide adenine dinucleotide phosphate), NADH (reduced nicotinamide adenine dinucleotide) and NADPH (reduced nicotinamide adenine dinucleotide phosphate), acetyl CoA, carboxylated biotin, S-adenosylmethionine, uridine diphosphate glucose. All of these molecules are carriers via a high energy linkage and, on release from the sequestration vehicle, can act to affect the processes or metabolism within the cell. Alternatively, these biologically active carriers may themselves provide the energy necessary for activation of the RNAi construct or RNAi core contained in the sequestration vehicle. For example, upon application of energy to the system, i.e., energy activation of the sequestration vehicle, release of the biologically activated carrier may undergo hydrolysis, reduction/oxidation or hydrogen exchange any of which may alter (increase or decrease) gene expression and therefore the RNAi machinery within the cell. Upon this trigger the RNAi constructs or RNAi cores formerly sequestered could exert their effects to a greater extent than without the signals provided by the biologically activated carriers. These biologically activated carriers in addition to being freely incorporated within a sequestration vehicle may also be chemically linked or bound to the sequestration vehicle itself to facilitate targeting within a cell, the cell surface, a tissue or organism. For example, a biologically activated carrier may be associated with the exterior or outer boundary of the sequestration vehicle in a manner whereby, upon introduction into the cell, tissue or organism, the energy of the high energy bond of the biologically activated carrier is released. This release could supply the energy for energy activation of the sequestration vehicle, thus liberating or releasing the RNAi constructs or RNAi cores which have been held inactive.

Modifications to the sequestration vehicles may also serve to functionalize the sequestration vehicle for stability, strength, or improvement of any physicochemical property or for therapeutic load.

### Pharmaceutical Compositions, Administration, and Dosing

The present invention also provides a pharmaceutical composition comprising a tripartite RNAi construct described herein and a pharmaceutically acceptable carrier or diluent. Pharmaceutical compositions of the present invention include, but are not limited to, solutions, emulsions, tablets, foams and liposome-containing formulations. The pharmaceutical compositions and formulations of the present invention may comprise one or more penetration enhancers, carriers, excipients or other active or inactive ingredients.

The pharmaceutical compositions of the present invention may be administered in a number of ways depending upon whether local or systemic treatment is desired and upon the area to be treated.

Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. RNAi constructs with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety.

The pharmaceutical formulations and compositions of the present invention may also include surfactants. The use of surfactants in drug products, formulations and in emulsions is well known in the art.

In one embodiment, the present invention employs various penetration enhancers to effect the efficient delivery of the RNAi constructs. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. One of skill in the art will recognize that formulations are routinely designed according to their intended use, i.e. route of administration.

Preferred formulations for topical administration include those in which the RNAi constructs of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). For topical or other administration, RNAi constructs of the invention may be encapsulated within liposomes or may form complexes thereto, in particular to cationic liposomes. Alternatively, oligonucleotides may be complexed to lipids, in particular to cationic lipids.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which tripartite RNAi constructs of the invention are administered in conjunction with one or more penetration enhancers surfactants and chelators. Preferred surfactants include fatty acids and/or esters or salts thereof, bile acids and/or salts thereof. Also preferred are combinations of penetration enhancers, for example, fatty acids/salts in combination with bile acids/salts. RNAi constructs of the invention may be delivered orally, in granular form including sprayed dried particles, or complexed to form micro or nanoparticles.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more tripartite RNAi constructs and one or more other chemotherapeutic agents which function by a non-RNAi mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bischloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents may be used individually (e.g., 5-FU and RNAi construct), sequentially (e.g., 5-FU and RNAi construct for a period of time followed by MTX and RNAi construct), or in combination with one or more other such chemotherapeutic agents (e.g., 5-FU, MTX and RNAi construct, or 5-FU, radiotherapy and RNAi construct).

Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Two or more combined compounds may be used together or sequentially.

The formulation of therapeutic RNAi construct containing-compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency can generally be estimated based on EC₅₀s found to be effective for in vitro and in vivo animal models.

### Diagnostics/Kits

The compounds of the present invention can be utilized for diagnostics, therapeutics, prophylaxis and as research reagents and kits. For use in kits and diagnostics, the RNAi constructs of the present invention, either alone or in combination with other compounds or therapeutics, can be used as tools in differential and/or combinatorial analyses to elucidate expression patterns of a portion or the entire complement of genes expressed within cells and tissues.

### Therapeutic Use

By inhibiting the expression of a gene, the RNAi constructs of the present invention can be used to treat any disease involving the expression of a protein. Examples of diseases that can be treated by the subject RNAi constructs, just to illustrate, include: cancer, retinopathies, autoimmune diseases, inflammatory diseases (i.e., ICAM-1 related disorders, Psoriasis, Ulcerative Colitus, Crohn's disease), viral diseases (i.e., HIV, Hepatitis C), miRNA disorders, and cardiovascular diseases.

In one embodiment, in vitro treatment of cells with the subject RNAi constructs can be used for ex vivo therapy of cells removed from a subject (e.g., for treatment of leukemia or viral infection) or for treatment of cells which did not originate in the subject, but are to be administered to the subject (e.g., to eliminate transplantation antigen expression on cells to be transplanted into a subject). In addition, in vitro treatment of cells can be used in non-therapeutic settings, e.g., to evaluate gene function, to study gene regulation and protein synthesis or to evaluate improvements made to oligonucleotides designed to modulate gene expression or protein synthesis. In vivo treatment of cells can be useful in certain clinical settings where it is desirable to inhibit the expression of a protein. There are numerous medical conditions for which antisense therapy is reported to be suitable (see, e.g., U.S. Pat. No. 5,830,653) as well as respiratory syncytial virus infection (WO 95/22,553) influenza virus (WO 94/23,028), and malignancies (WO 94/08,003). Other examples of clinical uses of antisense sequences are reviewed, e.g., in Glaser. 1996. Genetic Engineering News 16:1. Exemplary targets for cleavage by oligonucleotides include, e.g., protein kinase Ca, ICAM-1, c-raf kinase, p53, c-myb, and the bcr/abl fusion gene found in chronic myelogenous leukemia.

The subject RNAi constructs can be used in RNAi-based therapy in any animal having RNAi pathway, such as human, non-human primate, non-human mammal, non-human vertebrates, rodents (mice, rats, hamsters, rabbits, etc.), domestic livestock animals, pets (cats, dogs, etc.), Xenopus, fish, insects (Drosophila, etc.), and worms (C. elegans), etc.

### EQUIVALENTS

Those skilled in the art will recognize, or be able to ascertain using no more than routine experimentation, many equivalents to the specific embodiments of the invention described herein. Such equivalents are intended to be encompassed by the following claims. The entire contents of all patents, published applications and other references cited herein are hereby expressly incorporated herein in their entireties by reference.

1. An RNAi construct comprising:
(a) an RNAi core comprising a blunt-ended double-stranded RNA (dsRNA) with or without a portion of said RNAi core substituted with a non-nucleic acid based functional moiety, said RNAi core consisting of a sense strand and an antisense strand, each strand being 25-30 nucleotides in length, wherein said sense strand is chemically modified, and wherein said antisense strand is at least partially complementary to and hybridizes with a transcript from a target gene, and,
(b) one or more terminal moieties, each independently selected from: a bimodal partner, a carrier mimic, a membrane intercalator, a lipophilic molecule, a reporter molecule, a vitamin, a drug, a toxin, a polymer, a peptide, an antibody or a functional fragment, a carbohydrate, a nucleic acid cleaving complex, a metal chelator, an intercalator, a crosslinking agent, a cholesterol, a lipid moiety, a phospholipid, biotin, phenazine, a folate, phenanthridine, anthraquinone, acridine, a fluorescein, rhodamine, a coumarin, a dye, an active drug substance, or a group that enhances a pharmacodynamic property selected from construct uptake, construct resistance to degradation, and/or sequence-specific hybridization with the transcript,
wherein the RNAi core is linked to said one or more terminal moieties either directly or through one or more linkers.

2. The RNAi construct of claim 1, wherein said bimodal partner is an antisense, a ribozyme, an RNAi molecule which antagonizes the function of a gene other than the target gene.

3. The RNAi construct of claim 1, wherein said sense strand is chemically modified by the incorporation of modified oligonucleotide backbones containing a phosphorous atom.

4-7. (canceled)

8. The RNAi construct of claim 1, wherein each strand of said blunt-ended dsRNA is 25-27 nucleotides in length.

9. (canceled)

10. The RNAi construct of claim 1, wherein a portion of said RNAi core is substituted with the functional moiety which imparts said RNAi construct a feature, property, or characteristic.

11. (canceled)

12. The RNAi construct of claim 10, wherein said blunt-ended dsRNA is 25 nucleotides in length, and wherein about 15-20 contiguous nucleotides of the dsRNA is retained while the remaining 5-10 nucleotides of the dsRNA are substituted with the functional moiety.

13. (canceled)

14. The RNAi construct of claim 1, wherein the functional moiety is a protein, a peptide, a carbohydrate, or a lipid.

15-20. (canceled)

21. The RNAi construct of claim 1, wherein said one or more terminal moieties comprise one or more chemical modifications, protecting groups, and/or substituent groups.

22-30. (canceled)

31. The RNAi construct of claim 1, wherein said terminal moieties are attached directly or via the linker to the RNAi core at a nucleobase position, a sugar position, or one of the terminal internucleoside linkages.

32-34. (canceled)

35. The RNAi construct of claim 1, wherein said linkers comprise a chain structure or an oligomer of repeating units.

36-39. (canceled)

40. The RNAi construct of claim 1, wherein said linkers non-covalently bind the RNAi core to the terminal moiety.

41-42. (canceled)

43. The RNAi construct of claim 1, further comprising a sequestration vehicle that carries, conveys, or holds inactive said RNAi construct.

44. The RNAi construct of claim 43, wherein said RNAi constructed is activated by an amount of energy applied to the sequestration vehicle.

45-49. (canceled)

50. The RNAi construct of claim 43, wherein said sequestration vehicle is modified for targeting.

51. (canceled)

52. The RNAi construct of claim 44, wherein said RNAi constructed is activated by the energy in either a spatial and/or temporal manner.

53-54. (canceled)

55. The RNAi construct of claim 43, wherein said sequestration vehicle has a modification on the 5′ end of the anti-sense strand, wherein said modification degrades in the presence of light with a particular wavelength.

56-57. (canceled)

58. A pharmaceutical composition comprising a RNAi construct of claim 1, and one or more pharmaceutically acceptable carriers, excipients, diluents, penetration enhancers, surfactants, and other active or inactive ingredients.

59-60. (canceled)

61. A pharmaceutical composition for injectable delivery of the RNAi construct of claim 1, and pharmaceutically acceptable carriers, excipients, diluents, penetration enhancers, surfactants, and other active or inactive ingredients for injection.

62. A method of modulating the expression of a target gene in a cell, tissue, or organism, comprising contacting the cell, tissue, or organism with the RNAi construct of claim 1, wherein the antisense strand is at least partially complementary to and hybridizes with a transcript from the target gene.

63-65. (canceled)

66. The method of claim 62, wherein the RNAi construct comprises a sequestration vehicle, and the method further comprising:
activating the RNAi construct via the application of energy from an energy source sufficient to trigger the release or activation of said RNAi construct from the sequestration vehicle.

67. (canceled)
